(19) United States
(12) Patent Application Publication
(10) Pub. No.: US 2017/0219592 A1
(43) Pub. Date: Aug. 3, 2017

(54) PREDICTION OF CANCER TREATMENT BASED ON DETERMINATION OF ENZYMES OR METABOLITES OF THE KYNURENINE PATHWAY

(71) Applicant: IMMUSMOL SAS, Pessac (FR)

(72) Inventor: Alban BESSEDE, Bordeaux (FR)

(73) Assignee: ImmuSmol SAS, Pessac (FR)

(21) Appl. No.: 15/328,669

(22) PCT Filed: Jul. 24, 2015
(86) PCT No.: PCT/EP2015/067063

§ 371 Date: Jan. 24, 2017

(30) Foreign Application Priority Data
Jul. 24, 2014 (GB) 1413162.7

(51) Int. Cl. G01N 33/574

(52) U.S. Cl. CPC G01N 33/57484; G01N 2800/52

(57) ABSTRACT

The present invention relates to a method of predicting the therapeutic efficacy of at least one therapy approach in the treatment of a neoplastic disease in a patient. The method comprises the following steps:
a) Determining the presence or concentration of at least one enzyme or metabolite of the Kynurenine pathway in a patient sample, and
b) Concluding, from step a), whether the at least one therapy approach will be therapeutically effective in the treatment of the neoplastic disease.

The present invention is related to the prediction of cancer treatment based on determination of enzymes or metabolites of the Kynurenine pathway

Cancer immunotherapy has recently emerged as an attractive approach to treat patients with durable tumor response. Several compounds, which aim at stimulating the anti-tumoral host immune response, are currently in clinical development and one being used in clinic for the treatment of metastatic melanoma—an antibody targeting Cytotoxic T lymphocyte antigen-4 (CTLA-4, Ipilimumab), which is a receptor involved in the immunological synapse. With the same rationale of inhibiting immune checkpoints, PD1/PDL-1 receptor/ligands axis is one of the most promising therapeutic strategies. Besides CTLA4 and PD1 axis, several other mechanisms are also involved in the so-called tumor immune escape (Ott et al. 2013).

However, even if long-term objective tumor response has been achieved with immunotherapy (e.g., adoptive transfer of engineered cytotoxic T lymphocytes, Interleukin-2, anti-CTLA4, anti PDL1), only of small fraction of the respective patient cohort achieves benefit from the treatment.

To date, only 30 to 35% of patients suffering from advanced melanoma will benefit from anti PD1 therapy (Topalian et al. 2012)—in other words, 65 to 70% of patients receive the treatment without benefit.

It is therefore critical to identify those patients that might benefit from such therapy. This would avoid side effects in non-responding patients, and, from an economical point of view, healthcare expenditures could be kept at bay by avoiding unnecessary treatments.

Tryptophan metabolism and the Kynurenine pathway are physiological mechanisms which aim at preserving immune homeostasis and tolerance to avoid acute and chronic hyper inflammatory response—as can be seen in cancer immunotherapy. This pathway is initiated by three different enzymes:

Indoleamine 2,3 dioxygenase isoform 1 (Ido1)
Indoleamine 2,3 dioxygenase isoform 2 (Ido2)
Tryptophan 2,3 dioxygenase (Tdo2).

These enzymes catabolize tryptophan to form L-Kynurenine the first stable metabolite of this pathway, which in turn is metabolized to a series of metabolites collectively known as kynurenines (see FIGS. 3a and 3b). Certain of these metabolites exert immunoregulatory properties, especially L-Kynurenine, 3-hydroxyAnthranilic Acid or Cinnabarinic acid. In particular, L-Kynurenine has been shown recently to limit anti-tumoral immune response in brain tumors (Opitz et al, 2012) but also to limit acute inflammatory response (Bessede et al, 2014).

### SUMMARY OF THE INVENTION

It is an object of the present invention to provide means of predicting the therapeutic efficacy of at least one therapy approach in the treatment of a neoplastic disease in a patient.

It is another object of the present invention to provide a method of treating a neoplastic disease which has a high likelihood of achieving a therapeutic effect.

It is another object of the present invention to provide patients suffering from a neoplastic disease, or practitioners treating them with a predictive tool that helps them to avoid side effects and ineffective treatments.

### EMBODIMENTS OF THE INVENTION

These and other objects are met with methods and means according to the independent claims of the present invention. The dependent claims are related to preferred embodiments. It is yet to be understood that value ranges delimited by numerical values are to be understood to include the said delimiting values.

### SUMMARY OF THE INVENTION

Before the invention is described in detail, it is to be understood that this invention is not limited to the particular component parts of the devices described or process steps of the methods described as such devices and methods may vary. It is also to be understood that the terminology used herein is for purposes of describing particular embodiments only, and is not intended to be limiting. It must be noted that, as used in the specification and the appended claims, the singular forms “a,” “an” and “the” include singular and/or plural referents unless the context clearly dictates otherwise. It is moreover to be understood that, in case parameter ranges are given which are delimited by numeric values, the ranges are deemed to include these limitation values.

According to one embodiment of the invention a method of predicting the therapeutic efficacy of at least one therapy approach in the treatment of a neoplastic disease in a patient is provided, which method comprises the following steps:
a) Determining the presence or concentration of at least one enzyme or metabolite of the Kynurenine pathway in a patient sample, and
b) Concluding, from step a), whether the at least one therapy approach will be therapeutically effective in the treatment of the neoplastic disease.

According to another embodiment of the invention a method of treating a neoplastic disease in a patient is provided, which method comprises the following steps:
a) Determining the presence or concentration of at least one enzyme or metabolite of the Kynurenine pathway in a patient sample, and
b) Dependent on the result of step a), applying, in the patient, one or more therapy approaches.

According to another embodiment of the invention Use of at least one therapy approach in the treatment of a neoplastic disease in a patient is provided, which use encompasses:
a) Determining the presence or concentration of at least one enzyme or metabolite of the Kynurenine pathway in a patient sample, and
c) Dependent on the result of step a), applying, in the patient, one or more therapy approaches.

Preferably, it is provided that the presence, absence or concentration of at least one enzyme or metabolite of the Kynurenine pathway is predictive for the efficacy of said therapy approach. Preferably, in said method
a) the presence or a high concentration of at least one enzyme or metabolite of the Kynurenine pathway is predictive for a good efficacy of said therapy approach, and/or
b) the absence or low concentration of at least one enzyme or metabolite of the Kynurenine pathway is predictive for a poor efficacy of said therapy approach.

Preferably, it is provided that the at least one therapy approach encompasses the administration of immunotherapy.

The terms “immunotherapy” and “immunotherapeutic”, as used herein, encompass all methodologies which aim at stimulating the immune system, especially against tumour antigens.

Immunotherapy is the treatment of disease by inducing, enhancing, or suppressing an immune response. Immunotherapies designed to elicit or amplify an immune response are classified as activation immunotherapies, while immunotherapies that reduce or suppress are classified as suppression immunotherapies. Activation immunotherapy encompasses the administration of Immunomodulators, Cell based Immunotherapies, Cancer immunotherapy, Dendritic cell-based immunotherapy, Dendritic cell-based immunotherapy, adoptive cell transfer, Autologous immune enhancement therapy, application of Genetically engineered T cells, Genetically engineered T cells and vaccines.

Some of these immunotherapy approaches encompass the administration of an immunotherapeutic drug.

Preferably, said immunotherapeutic drug is administered to the patient in one or more doses.

In a particularly preferred embodiment, said one or more doses of the immunotherapeutic drug are administered, to the patient, in a therapeutically effective amount, and in a pharmacologically acceptable formulation or galenic.

In a preferred embodiment it is provided that the therapy approach, or the immunotherapeutic drug, interferes with an immune checkpoint.

Immune checkpoints refer to a plethora of inhibitory pathways hardwired into the immune system that are crucial for maintaining self-tolerance and modulating the duration and amplitude of physiological immune responses in peripheral tissues in order to minimize collateral tissue damage. It is now clear that tumours co-opt certain immune-checkpoint pathways as a major mechanism of immune resistance, particularly against T cells that are specific for tumour antigens. Because many of the immune checkpoints are initiated by ligand-receptor interactions, they can be readily blocked by antibodies or modulated by recombinant forms of ligands or receptors. Cytotoxic T-lymphocyte-associated antigen 4 (CTLA4) antibodies were the first of this class of immunotherapeutics. Preliminary clinical findings with blockers of additional immune-checkpoint proteins, such as programmed cell death protein 1 (PD1), indicate broad and diverse opportunities to enhance antitumour immunity with the potential to produce durable clinical responses.

All these approaches, involve that the patient's immune system itself becomes active to attack the neoplastic disease. As the inventors surprisingly show, the concentration of enzymes and/or metabolites from the Kynurenine pathway in a patient sample are predictive for the therapeutic efficacy of therapies interfering with an immune checkpoint in the treatment of neoplastic diseases.

In another preferred embodiment it is provided that the therapy approach involves adoptive cell transfer.

Adoptive cell transfer refers to the transfer of cells, most commonly immune-derived cells, back into the same patient or into a new recipient host with the goal of transferring the immunologic functionality and characteristics into the new host. If possible, use of autologous cells helps the recipient by minimizing Graft-versus-Host-Disease (GVHD) issues. For isolation of immune cells for adoptive transfer, a phlebotomist draws blood into tubes containing anticoagulant and the PBMC (Peripheral Blood Mononuclear cells) cells are isolated, typically by density barrier centrifugation.

In T cell-based therapies, these cells are expanded in vitro using cell culture methods relying heavily on the immunomodulatory action of interleukin-2 and returned to the patient in large numbers intravenously in an activated state. Anti-CD3 antibody is commonly used to promote the proliferation of T cells in culture. Research into interleukin-21 suggests it may also play an important role in enhancing the efficacy of T cell based therapies prepared in vitro. An emerging treatment modality for various diseases is the transfer of stem cells to achieve therapeutic effect. Clinically, this approach has been exploited to transfer either immune-promoting or tolerogenic cells (often lymphocytes) to patients to either enhance immunity against viruses and cancer or to promote tolerance in the setting of autoimmune disease, such as Type I diabetes or rheumatoid arthritis. Cells used in adoptive therapy may be genetically modified using recombinant DNA technology to achieve any number of goals. One example of this in the case of T cell adoptive therapy is the addition of chimeric antigen receptors, or CARs, to redirect the specificity of cytotoxic and helper T cells.

All these approaches, again, involve that the patient's immune system itself becomes active to attack the neoplastic disease. As the inventors surprisingly show, the concentration of enzymes and/or metabolites from the Kynurenine pathway in a patient sample are predictive for the therapeutic efficacy of adoptive cell transfer approaches in the treatment of neoplastic diseases.

In another preferred embodiment it is provided that the immunotherapeutic drug is a modulator, inhibitor, antagonist and/or binder of CTLA4, OX40, PD1, PDL1, 1Lag3, B7-H3, B7-H4, IDO1, IDO2, TDO2 and/or TIM3.

In the following table, some agents that target immune-checkpoint pathways are shown in an exemplary fashion:

| | Antibody or Ig fusion | |
|---|---|---|
| Target Biological function | protein | State of clinical development |
| CTLA4Inhibitory receptor | Ipilimumab | FDA approved for melanoma, Phase II |
| | | and Phase III trials ongoing for multiple |
| | | cancers |
| | Tremelimumab | Previously tested in a Phase III trial of |
| | | patients with melanoma; not currently |
| | | active |
| PD1 Inhibitory receptor | MDX-1106 (also known | Phase I/II trials in patients with |
| | as BMS-936558) | melanoma and renal and lung cancers |
| | MK3475 | Phase I trial in multiple cancers |
| | CT-011 | Phase I trial in multiple cancers |
| | AMP-224* | Phase I trial in multiple cancers |
| PDL1 Ligand for PD1 | MDX-1105 | Phase I trial in multiple cancers |
| | Multiple mAbs | Phase I trials planned for 2012 |
| LAG3 Inhibitory receptor | IMP321** | Phase III trial in breast cancer |
| | Multiple mAbs | Preclinical development |
| B7-H3 Inhibitory ligand | MGA271 | Phase I trial in multiple cancers |
| B7-H4 Inhibitory ligand | | Preclinical development |
| TIM3 Inhibitory receptor | | Preclinical development |
| OX40 secondary | Anti-OX40 | Clinical Trials |
| costimulatory receptor | | |
| IDO1 Initial step of | Indoximod, | Clinical trials and R&D |
| Kynurenine pathway | INCB024360 | |
| TDO2 Initial step of | | R&D |
| Kynurenine pathway | | |

CTLA4 = cytotoxic T-lymphocyte-associated antigen 4;
LAG3 = lymphocyte activation gene 3;
PD1 = programmed cell death protein 1;
PDL = PD1 ligand;
TIM3 = T cell membrane protein 3,
OX40 aka CD134;
TDO2 = Tryptophan 2,3-dioxygenase;
IDO1 = Indoleamine 2,3-dioxygenase 1
*PDL2-Ig fusion protein;
**LAG3-Ig fusion protein.

In still another preferred embodiment it is provided that the immunotherapeutic drug is a cancer vaccine.

A cancer vaccine is a vaccine that treats existing cancer or prevents the development of cancer in certain high-risk individuals. Vaccines that treat existing cancer are known as therapeutic cancer vaccines. One approach to cancer vaccination is to separate proteins from cancer cells and immunize cancer patients against those proteins, in the hope of stimulating an immune reaction that could kill the cancer cells. Therapeutic cancer vaccines are being developed for the treatment of breast, lung, colon, skin, kidney, prostate, and other cancers.

Another approach to therapeutic anti-cancer vaccination is to generate the immune response in situ in the patient using oncolytic viruses. This enhances the anti-tumor immune response to tumor antigens released following viral lysis and provides an in situ patient specific anti-tumor vaccine as a result.

Several cancer vaccines are currently in development by companies such as, Sipuleucel, Aduro (GVAX), Advaxis (ADXS11-001, ADXS31-001, ADXS31-164); ALVAC-CEA vaccine; Avax Technologies [AC Vaccine]; Amgen (talimogene laherparepvec); Accentia Biopharmaceuticals (BiovaxID in phase III); Bavarian Nordic (PROSTVAC); Celldex Therapeutics (CDX110, CDX1307 and CDX1401); The Center of Molecular Immunology (CimaVax-EGF); CureVac (CV9104); Dendreon Corp (Neuvenge); Galena Biopharma (NeuVax); Generex Biotechnology (Ae-37); Geron Corporation (GRNVAC1); GlaxoSmithKline (vaccine for melanoma targeting MAGE-A3); Globelmmune (Tarmogens, GI-4000, GI-6207, GI-6301); Heat Biologics (ImPACT Therapy); Immatics biotechnologies (e.g. IMA901); Merck (Stimuvax); Oncotherapy Science (peptide vaccines); Panacela Labs (MOBILAN); Prima BioMed LTD (Cvac), and Scancell Holdings (SCIB1).

All these approaches, again, involve that the patient's immune system itself becomes active to attack the neoplastic disease. As the inventors surprisingly show, the concentration of enzymes and/or metabolites from the Kynurenine pathway in a patient sample are predictive for the therapeutic efficacy of cancer vaccines in the treatment of neoplastic diseases.

Preferably, the immunotherapeutic drug is at least one selected from the group consisting of
a monoclonal antibody (murine, chimeric, humanized, human)
a fragment or derivative thereof (e.g., Fab, Fab2, scFv)
a new antibody format
a fusion peptide comprising at least one domain capable of binding an enzyme and/or a metabolite of the kynurenine pathway
a antibody mimetic,
an aptamer, and/or
a small molecule antagonist.

The above list encompasses different classes of protein therapeutics, plus aptamers and small molecules.

As used herein, the term “monoclonal antibody (mAb)”, shall refer to an antibody composition having a homogenous antibody population, i.e., a homogeneous population consisting of a whole immunoglobulin, or a fragment or derivative thereof. Particularly preferred, such antibody is selected from the group consisting of IgG, IgD, IgE, IgA and/or IgM, or a fragment or derivative thereof.

As used herein, the term “fragment” shall refer to fragments of such antibody retaining, in some cases, target binding capacities, e.g.
a CDR (complementarity determining region)
a hypervariable region,
a variable domain (Fv)
an IgG heavy chain (consisting of VH, CH1, hinge, CH2 and CH3 regions)
an IgG light chain (consisting of VL and CL regions), and/or
a Fab and/or F(ab)₂.

As used herein, the term “derivative” shall refer to protein constructs being structurally different from, but still having some structural relationship to, the common antibody concept, e.g., scFv, Fab and/or F(ab)₂, as well as bi-, tri- or higher specific antibody constructs. All these items are explained below.

Methods for the production and/or selection of chimeric, humanised and/or human mAbs are known in the art. For example, U.S. Pat. No. 6,331,415 by Genentech describes the production of chimeric antibodies, while U.S. Pat. No. 6,548,640 by Medical Research Council describes CDR grafting techniques and U.S. Pat. No. 5,859,205 by Celltech describes the production of humanised antibodies. In vitro antibody libraries are, among others, disclosed in U.S. Pat. No. 6,300,064 by MorphoSys and U.S. Pat. No. 6,248,516 by MRC/Scripps/Stratagene. Phage Display techniques are for example disclosed in U.S. Pat. No. 5,223,409 by Dyax. Transgenic mammal platforms are for example described in US200302048621 by TaconicArtemis.

IgG, scFv, Fab and/or F(ab)₂ are antibody formats well known to the skilled person. Related enabling techniques are available from the respective textbooks.

As used herein, the term “Fab” relates to an IgG fragment comprising the antigen binding region, said fragment being composed of one constant and one variable domain from each heavy and light chain of the antibody

As used herein, the term “F(ab)₂” relates to an IgG fragment consisting of two Fab fragments connected to one another by disulfide bonds.

As used herein, the term “scFv” relates to a single-chain variable fragment being a fusion of the variable regions of the heavy and light chains of immunoglobulins, linked together with a short linker, usually serine (S) or glycine (G). This chimeric molecule retains the specificity of the original immunoglobulin, despite removal of the constant regions and the introduction of a linker peptide.

The term “new antibody formats” encompasses, for example bi- or trispecific antibody constructs, Diabodies, Camelid Antibodies, Domain Antibodies, bivalent homodimers with two chains consisting of scFvs, IgAs (two IgG structures joined by a J chain and a secretory component), shark antibodies, antibodies consisting of new world primate framework plus non-new world primate CDR, dimerised constructs comprising CH3+VL+VH, and antibody conjugates (e.g., antibody or fragments or derivatives linked to a toxin, a cytokine, a radioisotope or a label). This list is however not restrictive.

Further, the term also encompasses immunotoxins, i.e., heterodimeric molecules consisting of an antibody, or a fragment thereof, and a cytotoxic, radioactive or apoptotic factor. Such type of format has for example been developed by Philogen (e.g., anti-EDB human antibody L19, fused to human TNF), or Trastuzumab emtansine (T-DM1), which consists of trastuzumab linked to the cytotoxoic Mertansine (DM1).

As the inventors of the present invention have shown that 3HAA is overabundant in tumor tissue, targeting 3HAA with a specific immunotoxin represents a very promising therapeutic approach of site-directed tumor therapy.

The term “fusion peptide” or “fusion protein” relates, for example, to proteins consisting of an immunoglobulin Fc portion plus a target binding moiety (so-called-cept molecules).

The term “antibody mimetic” relates to target binding proteins which are not related to immunoglobulins. Many of the above mentioned techniques, like phage display, are applicable for these molecules as well. Such antibody mimetics are for example derived from Ankyrin Repeat Proteins, C-Type Lectins, A-domain proteins of Staphylococcus aureus, Transferrins, Lipocalins, Fibronectins, Kunitz domain protease inhibitors, Ubiquitin, Cysteine knots or knottins, thioredoxin A, and so forth, and are known to the skilled person in the art from the respective literature.

The term “aptamer”, as used herein, relates to nucleic Acid species, which are capable of binding to molecular targets such as small molecules, proteins, nucleic Acids, and even cells, tissues and organisms. Aptamers are useful in biotechnological and therapeutic applications as they offer molecular recognition properties that rival that of the commonly used biomolecule, antibodies. In addition to their discriminate recognition, aptamers offer advantages over antibodies or other target binders as they can be engineered completely in a test tube, are readily produced by chemical synthesis, possess desirable storage properties, and elicit little or no immunogenicity in therapeutic applications. Aptamers can for example be produced through repeated rounds of in vitro selection or equivalently, SELEX (systematic evolution of ligands by exponential enrichment) to bind

The term “small molecule antagonist”, as used herein, relates to a low molecular weight organic compound, which is by definition not a polymer. The term small molecule, especially within the field of pharmacology, is usually restricted to a molecule that also binds with high affinity to a biopolymer such as protein, nucleic Acid, or polysaccharide and in addition alters the activity or function of the biopolymer. The upper molecular weight limit for a small molecule is often set at 800 Daltons, which allows for the possibility to rapidly diffuse across cell membranes so that they can reach intracellular sites of action. In addition, this molecular weight cutoff is a necessary but insufficient condition for oral bioavailability. Small molecules acting as antagonists against a given target, e.g., an enzyme and/or a metabolite of the kynurenine pathway, can be found by high throughput screening of respective libraries comprising a large variety of different small molecular candidates.

As used herein, the term “kynurenine pathway” encompasses enzymes and metabolites of said pathway.

In a preferred embodiment, said enzyme of the kynurenine pathway is at least one selected from the group consisting of Kynurenine formamidase, Kynurenine amino-transferase, Kynurenine 3-hydroxylase (also called Kynurenine mono-oxygenase), Kynureninase (also called L-Kynurenine hydrolase), Kynurenine amino-transferase, 3-Hydroxyanthranilic Acid oxygenase (also called 3-Hydroxanthranilate dioxygenase), indoleamine 2,3 dioxygenase 1 (IDO1), indoleamine 2,3 dioxygenase 2 (IDO2), and/or tryptophan 2,3 dioxygenase 2 (TDO2).

In another preferred embodiment, said metabolite of the kynurenine pathway is at least one selected from the group consisting of L-Tryptophane, N-Formylkynurenine, D and/or L-Kynurenine, Kynurenic acid, Quinaldic acid, Kynuramine, 3-hydroxy-L-kynurenine, 3-hydroxy-D-kynurenine, Xanthommatin, Anthranilic Acid, Xanthurenic Acid, 3-Hydroxy Anthranilic Acid, Picolinioc Acid and/or Quinolinic Acid and/or Cinnabarinic Acid.

An overview of particularly preferred enzymes and metabolites of the Kynurenine pathway is shown in FIG. 3.

In a preferred embodiment of the invention, the method or use encompasses, in step a), the determination of the concentration of two or more enzymes or metabolites of the Kynurenine pathway, whereupon a logical or arithmetical operation is made based on the determined concentrations, the result of which is then taken as a basis for the decision made in step b). This embodiment encompasses different sub-embodiments. In one preferred embodiment (the “multiplex” embodiment), two or more enzymes or metabolites of the Kynurenine pathway are detected simultaneously in the same sample. This requires—in some embodiments that different immunoligands are used which bind to the different enzymes, or metabolite-carrier complexes, Preferably, the different detection immunoligands are labelled with different labels (e.g., different fluorophores that have different excitation/emission wavelengths) so that the abundance of the different enzymes or metabolites of the Kynurenine pathway can be determined individually. In another preferred embodiment (the “parallel” embodiment), two or more enzymes or metabolites of the Kynurenine pathway are detected simultaneously in different subsamples. This requires that the sample to be investigated is subdivided into different subsamples, or different aliquots are drawn from the sample, i.e., one subsample or aliquot for each enzyme or metabolites of the Kynurenine pathway. The subsamples or aliquots are then investigated as described.

The values obtained in the quantification of individual enzymes or metabolites of the Kynurenine pathways can be combined for the purpose of disease assessment, e.g., by forming an arithmetical or logical operation on the determine concentrations. Such multi-parametric analysis, can provide better information with respect to a given prediction. This approach further allows the formation of a molecular signature for a given disease.

In another preferred embodiment of this approach, the detection of one or more enzymes or metabolites of the Kynurenine pathway is combined with the detection of one or more other analytes, e.g., a hormone receptor, to further improve the specificity of a diagnosis, prediction or prognosis.

Preferably, the arithmetical operation is at least one selected from the group consisting of a summation, multiplication, quotient, and/or ratio.

According to another embodiment of the invention, the presence or concentration of at least one enzyme or metabolite in the patient sample is determined by at least one method selected from the group consisting of
Immunohistochemistry, ELISA, EIA and/or Immunofluorescence
in situ PCR (e.g., in tissue slices)
realTime PCR (also called rT PCR, or quantitative PCR) (e.g., in homogenized/liquid samples)
Gas Chromatography/Mass Spectroscopy (GC/MS)
High Performance Liquid Chromatography (HPLC)
Liquid Chromatography/Mass spectroscopy (LC/MS)
Fluorescence-activated cell sorting (FACS)

Preferably, the metabolite of the kynurenine pathway in the sample is derivatized, prior to detection, by conjugating it to a carrier molecule.

In such way, a metabolite-carrier complex is created. Preferably, such carrier molecule is a protein or oligopeptide. Preferably, these carrier molecules have a minimum size of at least 1000 Da, more preferably 5000 Da. Further, these carrier molecules carry functional groups like amino groups and/or carboxylic groups, which make them accessible to binding to Kynurenine pathway metabolites by means of appropriate derivatization.

Preferably, the kynurenine pathway metabolites are bound to the carrier molecule by means of at least one coupling agent.

Preferably, these coupling agents promote the formation of amide bonds or peptide bonds, preferably bonds in which a carboxylic function of one entity and an amide function of another entity is involved.

In a preferred embodiment of the invention, a carbodiimide coupling agent is used, which is preferably one selected from the group consisting of
1-Ethyl-3-(3-dimethylaminopropyl) Carbodiimide Hydrochloride (EDC)
1-Cyclohexyl-3-(2-morpholinoethyl) carbodiimide (CMC)
N,N′-Dicyclohexylcarbodiimide (DCC)
Diisopropylcarbodiimide (DIC)

Carbodiimides are not traditional crosslinkers in that the crosslinker (i.e., the coupling agent) itself does not become part of the protein-protein complex. Carbodiimides instead covalently link two moieties directly together by forming an amide bond between a carboxylic acid group of one moiety (e.g., the analyte) and an amine group of another (e.g., the carrier protein). Because of the mechanism of carbodiimide crosslinkers, they are by nature zero length, i.e., they do not become part of the molecule, and heterobifunctional crosslinkers.

1-Ethyl-3-(3-dimethylaminopropyl)carbodiimide (EDC) is a water soluble carbodiimide usually obtained as the hydrochloride. It is typically employed in the 4.0-6.0 pH range. It is generally used as a carboxyl activating agent for the coupling of primary amines to yield amide bonds, or to activate corboxyl groups for the coupling to amine groups to yield peptide bonds.

According to another example, 3HAA can be bound to a protein carrier, like BSA, with ethyl chloroformate, which can be used to establish an amide bond between the carboxylic group of 3HAA and a free NH2 group of the protein carrier, e.g., at the N-terminus or at a side chain of lysine residues.

As another example, L-Kynurenine can be activated with the carbodiimide 1-Ethyl-3-(3-dimethylaminopropyl) Carbodiimide Hydrochloride to establish an amide bond between a carboxylic group of L-Kynurenine and a free NH2 group of the protein carrier, e.g., at the N-terminus or at a side chain of lysine residues.

Other approaches are available for conjugating small molecular analytes to antigenic carriers, e.g., carrier proteins. The choice of which conjugation chemistry to use depends on the functional groups available on the small molecular analytes, the required orientation, and the possible effect of conjugation on biological and antigenic properties.

For example, proteins and peptides have primary amines (the N-terminus and the side chain of lysine residues), carboxylic groups (C-terminus or the side chain of aspartic Acid and glutamic Acid), and sulfhydryl groups (side chain of cysteine residues) that can be targeted for conjugation. Preferably, one or more of the many primary amines in a carrier protein are used to couple a small molecular analyte.

Preferably, in the method according to the invention, it is provided that, in case the small molecular analyte to be bound to the carrier comprises, in its native state, an amine group, the method further comprises, prior to the step of derivatizing the analyte, a step of induced chemical transformation of said amine group to obtain a carboxyl group.

Such chemical transformation can for example be effected by use of anhydrides. Anhydrides can acylate amine groups and thus convert the amine functionality to a carboxyl group. For example, succinic, glutaric, maleic or citraconic anhydride can be used for this purpose. Subsequently, the thus-obtained carboxylic group can be used to couple the small molecular analyte to the carrier, and subsequent detection thereof by an immunoligand in the method according to the invention.

Preferably, in the method according to the invention, it is provided that, in case the small molecular analyte to be bound to the carrier comprises, in its native state, a sulfhydryl group, wherein the method further comprises, prior to the step of derivatizing the analyte, a step of induced chemical transformation of said sulfhydryl group to obtain a carboxyl group.

Such chemical transformation can for example be effected by modification with BMPA (N-β-maleimidopropionic acid). The maleimide function of the latter will spontaneously react covalently with the sulfhydryl group, and the rest of the BMPA molecule will then display the carboxylic group that forms part of BMPA. Subsequently, the thus-obtained carboxylic group can be used to couple the small molecular analyte to the carrier, and subsequent detection thereof by an immunoligand in the method according to the invention.

Preferably, in the method according to the invention, it is provided that, in case the small molecular analyte to be bound to the carrier comprises, in its native state, a hydroxyl group, wherein the method further comprises, prior to the step of derivatizing the analyte, a step of induced chemical transformation of said sulfhydryl group to obtain a carboxyl group.

Such chemical transformation can for example be effected by modification with chloroacetic acid. The reaction occurs under basic conditions leading to the formation of an ether bond, and the rest of the chloroacetic acid molecule will then display its carboxylic group. Subsequently, the thus-obtained carboxylic group can be used to couple the small molecular analyte to the carrier, and subsequent detection thereof by an immunoligand in the method according to the invention.

It is evident from these examples that the chemical transformation can encompass both the actual chemical modification of the respective functional group into a carboxylic group, as well as the use of a bifunctional adaptor molecule, part of which binds to the functional group in such way that an carboxylic group of said bifunctional molecule is displayed, and thus available for derivatization and subsequent coupling to the carrier, e.g., by means of the above mentioned carbodiimides.

The above approach provides an efficacious alternative to another preferred embodiment mentioned elsewhere, which provides that the small molecular analyte to be bound to the carriers comprises at least one amine group. This means that, in case of an amide or peptide bound formed, the carrier needs to provide a carboxyl group.

Preferably, a carrier molecule is used that is inherent to the sample. In this embodiment, naturally occurring carrier molecules are used, e.g., different serum proteins that are part of the sample.

Naturally occurring serum or blood proteins are for example Albumins, Globulins, Fibrinogens, Regulatory proteins or Clotting factors, and in particular Prealbuminm Alpha 1 antitrypsin, Alpha 1 acid glycoprotein, Alpha 1 fetoprotein, alpha2-macroglobulin, Gamma globulins, Beta 2 microglobulin, Haptoglobin, Ceruloplasmin, Complement component 3, Complement component 4, Lipoproteins, C-reactive protein (CRP), Lipoproteins (chylomicrons, VLDL, LDL, HDL), Transferrin, Prothrombin, MBL or MBP and naturally occurring mixtures thereof.

Naturally occurring proteins in other body fluids that can act as a sample are, for example,

(i) saliva proteins, like mucopolysaccharides and glycoproteins, α-amylase, lingual lipase, kallikrein, bradykinin, lysozame, lactoperoxidase, lactoferrin, immunoglobulin A, proline-rich proteins
(ii) urine proteins, like bilirubin, albumin, α2u-globulins, immunoglobulins A and M, and other proteins that are associated with proteinuria
(iii) seminal plasma proteins, like prealbumin, albumin, globulin, transferring, α-antitrypsin, β-lipoprotein, β-glycoprotein, orsomucoid, kininogen, peptide hormones, IgG, IgA and IgM

According to another preferred embodiment, the at least one carrier molecule is a carrier molecule that is added to the sample.

In this embodiment, a defined carrier molecule can be added to the sample in defined quantities, thus creating standardized conditions. Preferably, prior to adding the carrier molecule to the sample, the sample is deproteinized, in order to remove or at least denaturated all protein which is in the sample, and thus to support the application of standardized conditions.

Deproteinization can be carried out with standard methods known in the art, e.g, by use of tungstic acid, trichloracetic acid (TCA), perchloric acid (PCA) or metaphosphoric acid, followed by neutralization. Other approaches involve a combination of pH adjustment, and heating, or the use of protein adsorption on a column, gel filtration chromatography, as well as a mixture of the aforementioned approaches.

Preferably, the carrier molecule is at least one selected from the group consisting of:
keyhole limpet hemocyanin (KLH), or modified forms thereof
Albumins, like bovine serum albumin (BSA), or modified forms thereof
Blue Carrier* Protein, or modified forms thereof
Globulins, like Thyroglobulin, or modified forms thereof
soybean trypsin inhibitor, or modified forms thereof, and/or
muramyl dipeptide and derivatives, or modified forms thereof.

Most of these carrier molecules are proteins which provide primary amines as substrates for covalent attachment of Kynurenine pathway metabolites (in particular those having a carboxyl group) using a variety of crosslinking techniques (e.g., carbodiimides).

The term “modified forms” alludes to chemically modified variants of the respective carrier, like, e.g., ethylendiamine-modified BSA. The skilled person would readily understand how such concept of modified forms translates to other antigenic carriers. Anyway, the two most commonly used carriers are keyhole limpet hemocyanin (KLH) and bovine serum albumin (BSA).

Keyhole limpet hemocyanin (KLH) is the most widely used carrier protein. The copper-containing polypeptide belongs to a group of non-heme proteins called hemocyanins, which are found in arthropods and mollusks. KLH is isolated from keyhole limpets (Megathura crenulata).

Because KLH is from a class of proteins and a group of organisms that are evolutionarily distant from mammals, it is very “foreign” to the mammalian immune system. The protein is also highly immunogenic because of its very large size and complex structure. The molecule is composed of 350 kDa and 390 kDa subunits that associate to form aggregates ranging from 0.5 to 8 million daltons.

Each KLH protein molecule contains several hundred surface lysine groups that provide primary amines as substrates for covalent attachment of Kynurenine pathway metabolites (in particular those having a carboxyl group) using a variety of crosslinking techniques (e.g., carbodiimides). These features make KLH an extremely immunogenic and effective carrier protein for immunogen preparation. Although the large protein is sometimes difficult to work with because it has limited solubility, the commercial availability of stabilized and pre-activated formulations make it convenient to use.

Blue Carrier* Protein is a purified preparation of Concholepas concholepas hemocyanin (CCH). The large protein exhibits most of the same immunogenic properties as the popular carrier protein, keyhole limpet hemocyanin (KLH). However, its better solubility provides greater flexibility in immunogen preparation protocols by allowing a broader range of buffer and pH conditions for coupling Kynurenine pathway metabolites using crosslinking methods. The CCH protein is composed of two very large polypeptide subunits (404 and 351 kDa) that form an extremely stable heterodidecameric structure even in the absence of divalent cations. (By contrast, KLH has a less stable and soluble homodidecameric structure). The complex molecular arrangement of CCH subunits contains diverse repeated antigenic structures that elicit a strong immune reaction mediated by T and B lymphocytes.

Because of their large size and molecular complexity, KLH and CCH hemocyanins are carrier proteins of choice for use as immunogens to produce antibodies against Kynurenine pathway metabolites. Moreover, studies suggest that the strong DTH immune response elicited by hemocyanins in animals and in humans may have beneficial therapeutic effects in certain types of cancer. New developments in the immunotherapy of cancer have taken advantage of the unique immunogenic properties of hemocyanins in the development of novel conjugate vaccines for treatment of emerging diseases.

Bovine serum albumin (BSA; 67 kDa) belongs to the class of serum proteins called albumins. Albumins constitute about half the protein content of plasma and are quite stable and soluble. BSA is much smaller than KLH but is nonetheless fully immunogenic. It is a popular carrier protein for weakly antigenic compounds. BSA exists as a single polypeptide with 59 lysine residues, 30 to 35 of which have primary amines as substrates for covalent attachment of Kynurenine pathway metabolites (in particular those having a carboxyl group) using a variety of crosslinking techniques (e.g., carbodiimides).

Cationized bovine serum albumin (cBSA) is prepared by modifying native BSA with excess ethylenediamine, essentially capping all negatively-charged carboxyl groups with positively-charged primary amines. The result is a highly positively-charged protein (pI>11) that has significantly increased immunogenicity compared to native BSA. In addition, the increased number of primary amines provides for a greater number of antigen molecules to be conjugated with typical crosslinking methods.

Another suitable carrier is Ovalbumin (OVA; 45 kDa). Also known as egg albumin, ovalbumin constitutes 75% of protein in hen egg whites. OVA contains 20 lysine groups and is most often used as a secondary (screening) carrier rather than for immunization, although it is somewhat immunogenic. The protein also contains 14 aspartic Acid and 33 glutamic Acid residues that afford carboxyl groups. These groups can be used as targets for conjugation with Kynurenine pathway metabolites. Ovalbumin exists as a single polypeptide chain having many hydrophobic residues and an pI of 4.63. The protein denatures at temperatures above 56° C. or when subject to electric current or vigorous shaking. OVA is unusual among proteins in being soluble in high concentrations of the organic solvent DMSO, enabling conjugation to Kynurenine pathway metabolites that are not easily soluble in aqueous buffers.

Other suitable carriers are bovine thyroglobulin, or soybean trypsin inhibitor. Yet another suitable carrier is Muramyl dipeptide (Acetylmuramyl-Alanyl-Isoglutamine (NAc-Mur-L-ala-D-isoGln), or derivatives thereof, like Murabutide (NAcMur-L-Ala-D-Gln-alpha-n-butyl-ester). Muramyl dipeptide is a peptidoglycan constituent of both Gram positive and Gram negative bacteria. It is composed of N-acetylmuramic Acid linked by its lactic Acid moiety to the N-terminus of an L-alanine D-isoglutamine dipeptide. The immunization of a mammal with complexes of an antigen coupled to muramyl dipeptide enhances the immune response. Other suitable carriers encompass multi-poly (DL-alanine)-poly(L-lysine).

Some of the approaches set forth above require the use of a detection immunoligand to detect the presence and/or concentration of the enzyme or metabolite of the Kynurenine pathway.

In another preferred embodiment of the invention the detection immunoligand has been created against a complex consisting of a metabolite of the Kynurenine pathway and a carrier.

Preferably, such complex is identical to the metabolite-carrier complex that is being made as set forth above. This means, e.g., not only that the carrier can be the same, respectively, but also that the crosslinking chemistry can be the same (e.g., use of the same activator, e.g., a carbodiimide-based activator)

It is preferably provided that the detection immunoligand that specifically binds to the metabolite-carrier complex has been created by a method which comprises the following steps:
a) conjugating the Kynurenine pathway metabolite, in isolated form, to a carrier molecule to obtain an immunogenic conjugate,
b) carrying out an immunization experiment with said immunogenic conjugate, and
c) obtaining, directly or indirectly, detection antibodies from said experiment that specifically bind to the metabolite-carrier complex and/or to the metabolite.

As used herein, the term “carrier molecule” relates to a carrier to which a target molecule is bound in order to induce, in a host, an immune response against the target molecule. Preferably, the carrier is the same as is being used for derivatization of the analyte through the detection process. Said carrier may be one that does not elicit an immune response by itself either. However, the conjugate thus produced is immunogenic despite the low molecular weight of the Kynurenine pathway metabolite itself. Once the host immunized with the immunogenic conjugate has developed an immune response and generated antibodies against said conjugate, the metabolite-carrier complex or the Kynurenine pathway metabolite may also be recognized by the produced antibodies.

Such carrier protein can be, principally, any peptide or protein, preferably of a size above 1 kD, that can be coupled with any Kynurenine pathway metabolites. The carrier protein, because it is large and complex, confers immunogenicity to the conjugated Kynurenine pathway metabolite, resulting in production of antibodies against epitopes on the Kynurenine pathway metabolite, and/or the metabolite-carrier complex.

To create the best immunogen for this approach, it may be beneficial to prepare the conjugates with several different carriers and with a range of [Kynurenine pathway metabolite]: [carrier] coupling ratios.

Many proteins can be used as carriers and are chosen based on immunogenicity, solubility, and availability of useful functional groups through which conjugation with the Kynurenine pathway metabolite can be achieved.

Further, it is preferably provided that the immunization experiment comprises at least one step selected from the group consisting of:
Immunizing a mammal, obtaining spleen cells from said mammal and fusing them with immortalized cells to obtain antibody-producing hybridoma cells, and/or
Immunizing peripheral blood mononuclear cells which have been obtained from a mammal in vitro.

The first approach is known as the Köhler/Milstein technique, which has for the first time been described in Köhler & Milstein (1975).

This approach works by fusing myeloma cells with spleen cells from a mammal (preferably a mouse) that has been immunized with the above discussed target-carrier construct. Polyethylene glycol can be used to fuse adjacent plasma membranes of both cell types. In order to select hyridoma cells, a selective medium in which only fused cells can grow is used.

This can for example be achieved by exposing cells to aminopterin, which is a folic acid analogue that inhibits dihydrofolate reductase. Myeloma cells have lost the ability to synthesize hypoxanthine-guanine-phosphoribosyl transferase (HGPRT), an enzyme necessary for the salvage synthesis of nucleic acids. However, these cells can tackle the absence of HGPRT unless the de novo purine synthesis pathway is also disrupted. Exposure to aminopterin blocks the de novo pathway and makes myeloma cells fully auxotrophic for nucleic acids requiring supplementation to survive.

Unfused myeloma cells can thus not grow in an aminopterin containing medium, while unfused spleen cells cannot grow indefinitely because of their limited life span. Only fused hybrid cells, referred to as hybridomas, are able to grow indefinitely in such medium, because the spleen cell partner supplies HGPRT and the myeloma partner has traits that make it immortal.

This mixture of hybridoma cells is then diluted, and clones are grown from single parent cells on multi-well plates. The antibodies secreted by the different clones are then assayed for their ability to bind to the antigen with a suitable assay, such as ELISA, Antigen Microarray Assay, or immuno-dot blot. The most productive and stable clone is then selected for future use.

The second approach is also known as “in vitro immunization”, and consists, essentially, of immunizing peripheral blood mononuclear cells (PBMC). These can be first treated with 1-leucyl-1-leucine methyl ester (LLME) to remove suppressive cells, and are then immunized with the above discussed target-carrier construct, preferably in the presence of several cytokines and muramyl dipeptide (MDP).

PBMC thus treated can then be transformed with Epstein-Ban virus (EBV), and fused with mouse-human hetero myeloma host cells, to create EBV-immortalized B cell hybridomas. To efficiently expand antigen-specific B cells in the in vitro-immunized PBMC, cytokines such as IL-2 and IL-4 can be added. Further, CpG oligonucleotides can be used as adjuvants for inducing antigen-specific responses.

An example for such in vitro immunization approach is described in Tamura et al (2007).

In another preferred embodiment, it is provided that the mammal used for immunization, or from which the PMBC have been obtained, is transgenic with respect for at least part of their immunoglobulin gene loci.

This approach encompasses the use of a transgenic mammal (e.g., a rabbit, or a mouse) whose native immunoglobulin gene loci (e.g., Ig-heavy chain and Igκ-light chain loci) have been disrupted and which have transgenes encoding genes for human Immunoglobulin (see, for example, Lonberg et al. (1994). More preferably, the expression of more V gene segments by the transgenic mammal is provided, as described in Lonberg (2005), thereby expanding the potential repertoire of the recovered antibodies.

Transgenic mammal platforms used for such purpose are for example described in US200302048621 by TaconicArtemis.

Antibodies thus obtained are fully human, i.e., they have no non-human sequences at all, and have thus a decreases risk of immunogenicity.

The details of an immunization experiment according to the above embodiment are demonstrated in the examples set forth below.

It can be preferred, in these embodiments, that the binding chemistry to create the immunogenic conjugate as set forth above (consisting of the Kynurenine pathway metabolite and the carrier molecule, which conjugate is then used in the immunization experiment to obtain the detection antibody) is the same as the binding chemistry that is actually used for derivatizing the Kynurenine pathway metabolite that is actually in the sample, and which is to be detected. Same applies for the carrier molecule actually used.

This means for example, that, preferably, both (i) in the immunization experiment as well as (ii) prior to the detection of the Kynurenine pathway metabolite an activator is used to crosslink the Kynurenine pathway metabolites to the carrier molecule, namely, e.g., EDC, CMC, DCC, DIC, Woodward's Reagent K, CDI, and/or ECF (see below)

This means further that, preferably, both (i) in the immunization experiment as well as (ii) prior to the detection of the Kynurenine pathway metabolite the same carrier molecule is used, namely, e.g., KLH, BSA, Blue Carrier* Protein, Globulins, like Thyroglobulin, soybean trypsin inhibitor, muramyl dipeptide and derivatives, or modified forms of these carriers (see above).

The latter is particularly preferred in combination with prior deprotonization of the sample as set forth elsewhere herein.

By using the same crosslinking chemistry and the same carrier molecule both (i) in the immunization experiment and (ii) prior to the detection of the Kynurenine pathway metabolite, a high degree of specificity and sensitivity is ensured in the detection method according to the invention, because the detection antibody that is used for detecting a given metabolite-carrier-complex has actually been made by immunization with the same metabolite-carrier-complex.

In another preferred embodiment, it is provided that the detection immunoligand has been created by a method which comprises the following steps:
a) exposing said analyte, or a metabolite-carrier complex to a library of immunoligands, and
b) screening said library for detection immunoligands that specifically bind to the metabolite-carrier complex and/or to the analyte.

Libraries of immunoligands are, for example, in vitro antibody libraries. These can be naïve or synthetic libraries, or combinations of both, depending on the source of the antibody repertoire used for the library generation, Naïve libraries are constructed from light and heavy chain repertoires isolated from non-immunised donors. For example, naive libraries consisting of the repertoire of human IgM genes isolated from peripheral blood lymphocytes (PBL) (Marks et al., 1991) and from bone marrow or tonsils (Vaughan et al., 1996) have been constructed.

(Semi-) synthetic libraries can be derived from unrearranged antibody genes of germline cells by cloning the CDR-containing gene segments of the different heavy and light chain families and rearrangement in vitro by PCR (e.g. Hoogenboom and Winter, 1992). Other (semi-) synthetic libraries have “targeted” diversity and consist solely of one or a few VH and VL frameworks and contain partially randomised CDR's. The diversity is introduced by PCRs with DNA-oligonucleotides having degenerated codons at desired positions. Further, in vitro antibody libraries are, among others, disclosed in U.S. Pat. No. 6,300,064 by MorphoSys and U.S. Pat. No. 6,248,516 by MRC/Scripps/Stratagene.

In a preferred embodiment of said method, the exposure and screening process is comprised in an in vitro display method or a high throughput screening method.

The term “in vitro display method” relates to methods in which individual members of an antibody library are displayed on a given entity, while the genetic information encoding said molecule is comprised in said entity. The members of said antibody library are then screened against an immobilized target, and those entities binding the target are then recovered, together with their displaying entity comprising the encoding information, for further analysis. Such methods are reviewed, e.g., in Bradbury et al (2011), and are thus well known to the skilled person.

The term “High-throughput screening” relates to library screening methods using robotics, data processing and control software, liquid handling devices, and sensitive detectors, in order to screen a given library of molecules on an assay plate format, usually based on optical detection. Such methods are, e.g, described by de Wildt et al (2000).

Preferably, the in vitro display method is at least one selected from the group consisting of
Phage display
E. coli display
Yeast display
Fungal display
Ribosome display
Retrocyte display

These and other techniques are all well known to the skilled person. The following table shows some third party patents related to phage display methods.

| Company | Technology | Alias name | Key IP right US | Key IP right EP |
|---|---|---|---|---|
| CAT (now | | Griffiths | U.S. Pat. No. 5,885,793 | EP0589877 |
| MedImmune) | | McCafferty | U.S. Pat. No. 5,969,108 | |
| Genetech | Monovalent | | U.S. Pat. No. 5,821,047 | EP0564531 |
| | phage display | | | |
| Dyax | | Ladner | U.S. Pat. No. 5,223,409 | EP0436597 |
| Biosite | “Omniclonal” | Dower | U.S. Pat. No. 5,427,908 | EP0527839 |
| Affitech | “MBAS” | Breitling | U.S. Pat. No. 6,387,627 | EP0547201 |
| Crucell | “MAbstract” | | U.S. Pat. No. 6,265,150 | none |
| BioInvent | “Biopanning” | Frendeus | US2006199219 | EP1535069 |
| MorphoSys | “Cys Display” | | U.S. Pat. No. 6,753,136 | EP1144607 |
| Haptogen (now | DNA-binding | | U.S. Pat. No. 7,312,074 | EP1009827 |
| Wyeth) | domain | | | |
| | extrusion | | | |
| | display | | | |
| | (“DBDx”) | | | |
| Molecular | Cotranslational | Plueckthun | none | EP1902131 |
| Partners | translocation | | | |
| | of fusion | | | |
| | polypeptides | | | |
| Research | IgG expressed in | Georgiou | WO2008067547 | |
| Development | periplasm | | | |
| Foundation | captured with | | | |
| | an Fc-binding | | | |
| | fusion protein | | | |
| | tethered to | | | |
| | inner | | | |
| | membrane | | | |

The next table shows some third party patents related to other display methods.

| Company | Technology | Alias name | Key IP right US | Key IP right EP |
|---|---|---|---|---|
| Optein (CAT) | Ribosome display | Kawasaki | U.S. Pat. No. 5,643,768 | EP0494955 |
| Univ. Texas | E. coli display | Georgiou | U.S. Pat. No. 5,348,867 | EP0746621 |
| Dade Behring | E. coli display | | | EP0603672 |
| Universiteit | Bacterial display | | U.S. Pat. No. 6,190,662 | EP0848756 |
| Gent | | | | |
| Abbott | Yeast display | Wittrup | U.S. Pat. No. 6,300,065 | EP1056883 |
| Novozymes | Fungal display | | U.S. Pat. No. 6,767,701 | EP1124949 |
| Evotec | Beads display | | U.S. Pat. No. 5,849,545 | EP0667960 |
| One Cell | Gel microdroplets | Weaver | U.S. Pat. No. 6,806,058 | EP1399580 |
| Systems | (In vitro compartmentalization) | | | |
| Gen Hospital | RNA puromycin | Szostak | U.S. Pat. No. 6,207,446 | EP0971946 |
| Corp | | | | |
| Affitech | Cell-based antibody selection | | none | EP1802980 |
| | (“CBAS”) | | | |
| Res Dev | Twin arginine translocation | Georgiou | US2003219870 | EP1487966 |
| Foundation | (TAT) mediated display | | | |
| 4-Antibody | Retrocyte display | | WO09109368 | |

Again, it can be preferred, in these embodiments, that the binding chemistry to create the metabolite-carrier complex that is used in the screening method is the same as the binding chemistry that is actually used for derivatizing the Kynurenine pathway metabolite that is actually in the sample, and which is to be detected. Same applies for the carrier molecule actually used.

This means for example, that, preferably, both (i) in the screening method as well as (ii) prior to the detection of the Kynurenine pathway metabolite an activator is used to crosslink the Kynurenine pathway metabolites to the carrier molecule, namely, e.g., EDC, CMC, DCC, DIC, Woodward's Reagent K, CDI, and/or ECF (see below)

This means further that, preferably, both (i) in the screening method as well as (ii) prior to the detection of the Kynurenine pathway metabolite the same carrier molecule is used, namely, e.g., KLH, BSA, Blue Carrier* Protein, Globulins, like Thyroglobulin, soybean trypsin inhibitor, muramyl dipeptide and derivatives, or modified forms of these carriers (see above).

According to a preferred embodiment of the method of the invention, the Kynurenine pathway metabolite to be bound to the carrier comprises at least one carboxyl group.

According to another preferred embodiment of the method of the invention, the Kynurenine pathway metabolite to be bound to the carrier does not comprise, in its native state, a carboxyl group, but undergoes an induced chemical transformation which then creates a carboxyl group. This “induced chemical translation” can either transform an existing functional group into a carboxyl group, or add a molecular entity to the analyte, e.g., by covalent bonding, which molecular entity itself carries such carboxyl group.

In other words: This definition encompasses Kynurenine pathway metabolite that have, in their native state, a carboxyl group, as well as those Kynurenine pathway metabolites which do not, but which undergo an induced chemical transformation which then creates a carboxyl group. In both cases, however, the carboxyl group then serves as the starting point for derivatization and subsequent coupling to the carrier, e.g., by means of a carbodiimide based coupling agent. See furtehr details below.

Preferably, the patient sample is a tissue sample and/or a liquid sample. Said tissue sample is for example a tissue slice, or a homogenized sample from a biopsy. Said liquid sample is for example a urine sample, saliva sample, blood serum sample, blood plasma sample, feces sample, sweat sample, swab sample, smear sample, a cell culture supernatant or the like. The term “neoplastic disease”, as used herein, refers to an abnormal state or condition of cells or tissue characterized by rapidly proliferating cell growth or neoplasm. In a more specific meaning, the term relates to cancerous processes, e.g., tumors and/or leukemias.

In a very preferred embodiment, said neoplastic disease is selected from the group consisting of
Colorectal cancer
Breast cancer
Melanoma, and/or
Glioma and other tumors of the central nervous system

### EXPERIMENTS AND FIGURES

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

FIG. 1: Progression of tumor volume in CT26 tumor bearing mice challenged or not with anti-CTLA4. Data represents mean tumor volume (in mm³) from 6 mice in each experimental setting. According to the literature, mice treated with anti-CTLA4 displayed a decrease in tumor progression.

FIG. 2: Quantification of L-Kynurenine in plasma from CT26 tumor bearing mice challenged with anti-CTLA4 using EIA (Enzyme immunoassay). Derivatized plasma samples were incubated with 3D4-F2 mAb (at 0.01 mg/ml) and an HRP-Kynurenine conjugate (Tracer, at 1 μg/mL) for 1.5 hour at 37° C. on a maxisporp plate coated with anti mouse IgG. Reaction was revealed using TetraMethylBenzidine (TMB). L-Kynurenine levels are correlated with tumor volume (mm³) at the date of sacrifice.

FIG. 3a: Entry reaction which initiates the kynurenine pathway (L-Tryoptophan->L-Formylkynurenine, but is not part thereof. The step is catalyzed by either a) Indoleamine 2,3-dioxygenase (IDO1) or b) Tryptophan 2,3-dioxygenase (TDO2). If one of the two is blocked, the reaction can still take place, while blocking both may have severe side effects.

FIG. 3b: Overview of the kynurenine pathway with its enzymes and metabolites. The enzymes are as follows: i) Kynurenine formamidase, a) Kynurenine amino-transferase, b) Kynurenine 3-hydroxylase (also called Kynurenine mono-oxygenase), c) Kynureninase (also called L-Kynurenine hydrolase), d) Kynurenine amino-transferase, e) Kynureninase (also called L-Kynurenine hydrolase), and f) 3-Hydroxyanthranilic Acid oxygenase (also called 3-Hydroxanthranilate dioxygenase).

The metabolites are as follows: L-Formylkynurenine, Kynuramine, L-Kynurenine, Kynurenic Acid, 3-hydroxyL-kynurenine, Anthranilic Acid, 3-hydroxyanthranilic Acid, Xanthurenic Acid, Quinaldic Acid, Picolinioc Acid and/or Quinolinic Acid.

Please note that some metabolites and enzymes of the Kynurenine pathway are not shown. This applies for example, for Niacin, which is formed out of Quinolinic Acid.

FIG. 4: Crosslinking reaction between a carboxylic acid group of one moiety (R1, e.g., the analyte or the carrier protein) and an amine group of another moiety (R2, e.g., the carrier protein or the analyte), as catalyzed by a carbodiimide. In this case, the latter is EDC (1-Ethyl-3-(3-dimethylaminopropyl)carbodiimide). See more explanations in the text.

FIG. 5: Kynurenine concentration in a mice colorectal cancer model according to the response to anti-CTLA4. See more explanations in the text.

### EXAMPLE 1: KYNURENINE LEVEL IS ASSOCIATED WITH CLINICAL RESPONSE TO ANTI-CTLA4

### 1. Experimental Procedure

### 1.1. Tumor Experiments and In Vivo Blockade of Anti-CTLA4.

BALB/c mice were implanted subcutaneously (s.c.) on the right flank with 5×10⁴ of CT26 cells (purchased from ATCC). One hundred μg of α-mouse α-CTLA-4 (clone 4F10) were administered intraperitoneally (i.p.), either 3, 6, and 9 days following CT26 inoculation and tumour size was monitored using caliper.

### 1.2. L-Kynurenine Quantification in Plasma Using Enzyme Immuno Assay

Twenty four (24) days after cells inoculation, mice were anesthetized using ketamine/xylazine and subjected to plasma collection by intracardiac puncture.

L-Kynurenine was then quantified in plasma using a novel enzyme immunoassay in which the analyte was conjugated to a carrier, in order to make it detectable by an antibody that has been made against the same conjugate. Like other small metabolites, L-Kynurenine alone is not immunogenic, which makes it difficult to create antibodies that are capable of binding isolated L-Kynurenine with sufficient specificity, e.g., to detect it. For this reason, in a first step, L-Kynurenine is coupled to a carrier protein, which confers immunogenicity to the latter, thus making it possible to raise antibodies against it.

In a second step, L-Kynurenine in the sample is also derivatized prior to exposure to the detection antibody created with the method above, to render it detectable by the latter.

Because in this example, the detection antibody was murine, all endogenous proteins in the sample had to be precipitated before L-Kynurenine was then coupled (“derivatized”) to a carrier. The carrier was in this case BSA, and the antibody used had been obtained by immunization of a mouse with a conjugate consisting of L-Kynurenine and BSA.

Briefly, 100 μl of plasma and standards solutions were precipitated using 25 μl of Trichloro acid acetic (TCA) 1N, vortexed and centrifuged (10,000 g, 10 minutes, 4° C.). 80 μl of supernatant were equilibrated with 20 μl of Tris buffer 1M, pH=9, supplemented with Bovine Serum Albumin (BSA) to achieve 5 g/L and subjected to derivatization therewith using 100 μl of carbodiimide (EDC) and N-HydroxySuccinimide solubilized in MES buffer (0.3M, pH=6.3) over a 1 hour period under agitation (400 rpm) at 37° C. In this process, L-Kynurenine and other metabolites are coupled to BSA. Details of the derivatization and the conjugation are disclosed in patent application No GB 13 22 538 the content of which is fully incorporated herein by reference. HRP-Kynurenine tracer and a murine anti-L-Kynurenine monoclonal antibody were added to the solution at a final concentration at 0.3 μg/ml and 1 μg/ml respectively.

The latter solution was applied by mean of 200 μl per well on a maxisorp ELISA plate previously coated with unconjugated anti-mouse IgG immunoglobulin that has been raised against a conjugate consisting of L-Kynurenine and BSA. The plate was incubated for 1.30 hour at 37° C. and reaction was revealed using Tetramethylbenzidine. Coloration was monitored at 450 nm with a spectrophotometer.

### 2. Results

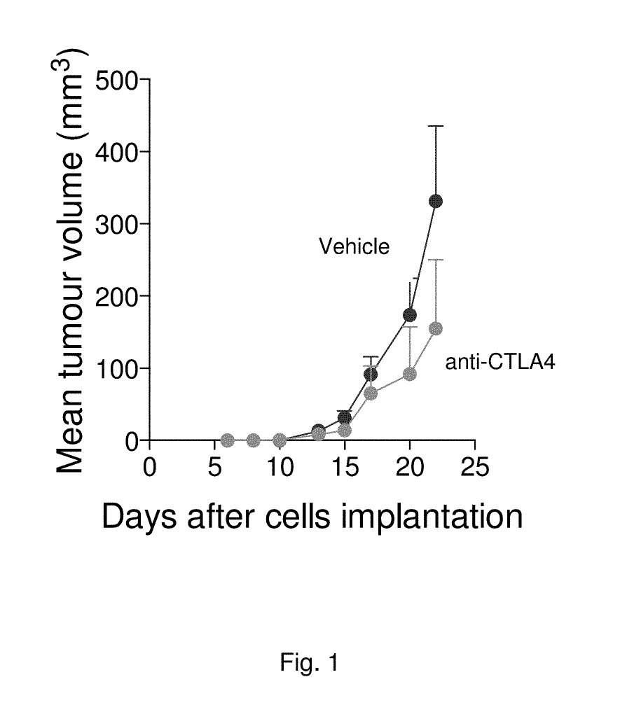

FIG. 1 shows the average tumour volume in CT26 tumor bearing mice treated with anti-CTLA4 (n=6) or the vehicle (n=6). Generally, a difference can be seen between the mice treated with anti-CTLA4 and with the vehicle.

However, in accordance with literature (Duraiswamy et al, Cancer Research, 2013), anti-CTLA4 exerts a benefit in only a fraction of the mice.

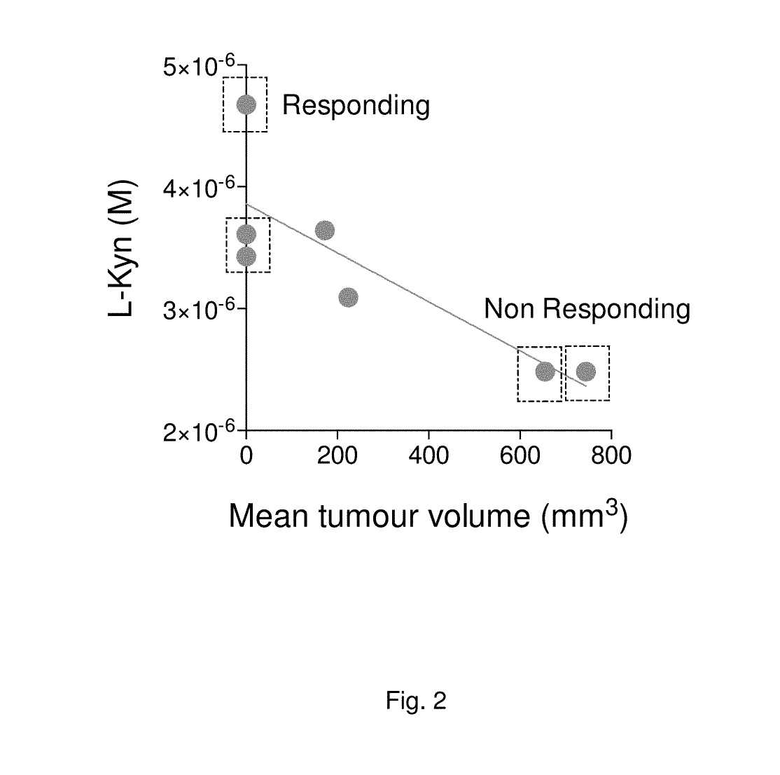

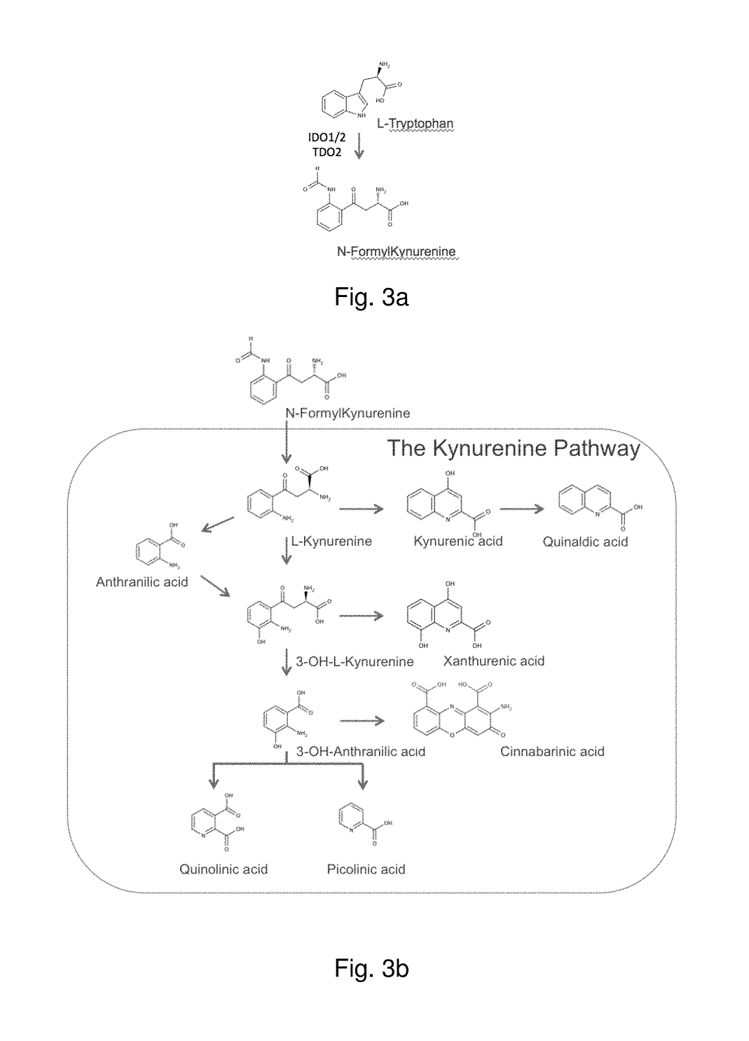

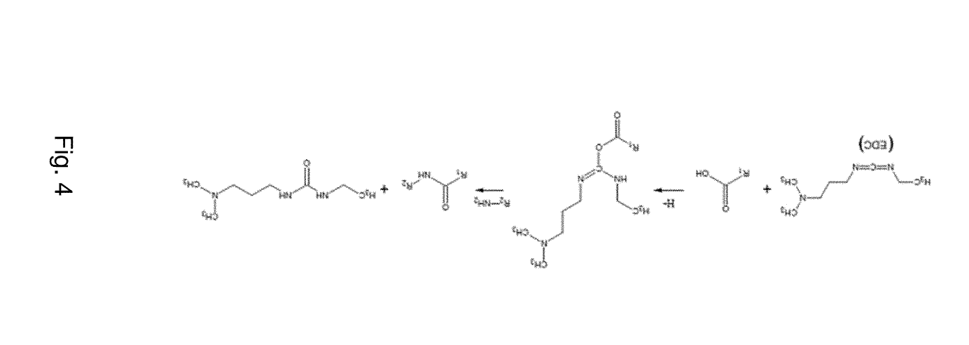

FIG. 2 shows a correlation between the L-Kynurenine plasma level and the tumour size from CT26 tumour bearing mice challenged with anti-CTLA4. These results indicate that mice with a higher plasma titer of L-Kynurenine display a better clinical response towards anti-CTLA4 than those with lower plasma titer.

The concentration of L-Kynurenine (which is a metabolite of the Kynurenine pathway) is thus predictive for the therapeutic efficacy of the drug anti-CTLA4 in the treatment of tumours.

### EXAMPLE 2: KYNURENINE/TRYPTOPHAN RATIO IS ASSOCIATED WITH CLINICAL RESPONSE TO ANTI-CTLA4

### 3. Experimental Procedure

### 3.1. Tumor Experiments and In Vivo Blockade of Anti-CTLA4.

BALB/c mice are implanted subcutaneously (s.c.) on the right flank with 5×10⁴ of CT26 cells (purchased from ATCC). One hundred μg of α-mouse α-CTLA-4 (clone 4F10) are administered intraperitoneally (i.p.), either 3, 6, and 9 days following CT26 inoculation and tumour size is monitored using caliper.

### 3.2. L-Kynurenine and Tryptophan Quantification in Plasma Using Immunoassay

Twenty four (24) days after cells inoculation, mice are anesthetized using ketamine/xylazine and subjected to plasma collection by intracardiac puncture.

L-Kynurenine is then quantified in plasma using a novel enzyme immuno assay in which the analyte is conjugated to a carrier, in order to make it detectable by an antibody that has been made against the same conjugate. Briefly, 100 μl of plasma and standards solutions are precipitated using 25 μl of Trichloro acid acetic (TCA) 1N, vortexed and centrifuged (10,000 g, 10 minutes, 4° C.). 80 μl of supernatant are equilibrated with 20 μl of Tris buffer 1M, pH=9 supplemented with Bovine Serum Albumin (BSA) to achieve 5 g/L and subjected to derivatization using 100 μl of carbodiimide (EDC) and N-HydroxySuccinimide solubilized in MES buffer (0.3M, pH=6.3) over a 1 hour period under agitation (400 rpm) at 37° C. Details of the derivatization and the conjugation are disclosed in patent application no GB 13 22 538, the content of which is fully incorporated herein by reference. HRP-Kynurenine tracer and a murine anti-L-Kynurenine monoclonal antibody are added to the solution at a final concentration at 0.3 μg/ml and 1 μg/ml respectively. The latter solution is applied by mean of 200 μl per well on a maxisorp ELISA plate previously coated with unconjugated anti-mouse IgG immunoglobulin. The plate is incubated for 1.30 hour at 37° C. and reaction is revealed using Tetramethylbenzidine. Coloration is monitored at 450 nm with a spectrophotometer.

Tryptophan is measured using commercially available ELISA kit—purchased from LDN, Nordhorn, Germany—according to the provider procedure.

### 4. Results

Experiments demonstrate that anti-CTLA4 therapy induced an increase in L-Kynurenine/Tryptophan ratio (as an indicator of IDO1, IDO2, TDO2 dependent tryptophan degradation) level in plasma compared to mice who received only vehicle.

Also, we show a correlation between the Kynurenine to tryptophan ratio in plasma and the tumour size from CT26 tumour bearing mice challenged with anti-CTLA4. These results indicate that mice with a higher plasma Kynurenine to Tryptophan ratio display a better clinical response towards anti-CTLA4 than those with lower ratio value.

The L-Kynurenine/Tryptophan ratio is thus predictive for the therapeutic efficacy of the drug anti-CTLA4 in the treatment of tumours.

### EXAMPLE 3: KYNURENINE LEVEL IS ASSOCIATED WITH CLINICAL RESPONSE TO ANTI-PD1

### 5. Experimental Procedure

### 5.1. Tumor Experiments and In Vivo Blockade of Anti-PD1.

BALB/c mice are implanted subcutaneously (s.c.) on the right flank with 5×10⁴ of CT26 cells (purchased from ATCC). Two hundred μg of α-mouse α-PD1 (clone RMP1-14) are administered intraperitoneally (i.p.), either 3, 6, and 9 days following CT26 inoculation and tumour size is monitored using caliper.

### 5.2. L-Kynurenine Quantification in Plasma Using Enzyme Immuno Assay

Twenty four (24) days after cells inoculation, mice are anesthetized using ketamine/xylazine and subjected to plasma collection by intracardiac puncture.

L-Kynurenine is then quantified in plasma using a novel enzyme immuno assay in which the analyte is conjugated to a carrier, in order to make it detectable by an antibody that has been made against the same conjugate. Briefly, 100 μl of plasma and standards solutions are precipitated using 25 μl of Trichloro acid acetic (TCA) 1N, vortexed and centrifuged (10,000 g, 10 minutes, 4° C.). 80 μl of supernatant are equilibrated with 20 μl of Tris buffer 1M, pH=9 supplemented with Bovine Serum Albumin (BSA) to achieve 5 g/L and subjected to derivatization using 100 μl of carbodiimide (EDC) and N-HydroxySuccinimide solubilized in MES buffer (0.3M, pH=6.3) over a 1 hour period under agitation (400 rpm) at 37° C. Details of the derivatization and the conjugation are disclosed in patent application No GB 13 22 538, the content of which is fully incorporated herein by reference. HRP-Kynurenine tracer and a murine anti-L-Kynurenine monoclonal antibody are added to the solution at a final concentration at 0.3 μg/ml and 1 μg/ml respectively.

The latter solution is applied by mean of 200 μl per well on a maxisorp ELISA plate previously coated with unconjugated anti-mouse IgG immunoglobulin. The plate is incubated for 1.30 hour at 37° C. and reaction is revealed using Tetramethylbenzidine. Coloration is monitored at 450 nm with a spectrophotometer.

### 6. Results

In accordance with literature (Duraiswamy et al, Cancer Research, 2013), our experiment shown a difference of the average tumor size between the mice treated with anti-PD1 and mice exposed only to the vehicle. However anti-PD1 exerts a benefit in only a fraction of the mice.

Also, we show a correlation between the L-Kynurenine plasma level and the tumour size from CT26 tumour bearing mice challenged with anti-PD1. These results indicate that mice with a higher plasma titer of L-Kynurenine display a better clinical response towards anti-PD1 than those with lower plasma titer.

The concentration of L-Kynurenine (which is a metabolite of the Kynurenine pathway) is thus predictive for the therapeutic efficacy of the drug anti-PD1 in the treatment of tumours.

### EXAMPLE 4: KYNURENINE LEVEL IN HUMAN SAMPLES IS ASSOCIATED WITH CLINICAL RESPONSE TO ANTI-CTLA4

### 7. Experimental Procedure

### 7.1. Clinical Settings and In Vivo Blockade of CTLA4.

Patients suffering from advanced metastatic melanoma are treated with Ipilimumab (fully human anti CTLA4, Bristol Myers Squibb, 3 mg/kg, i.v, over 90 mins, triweekly, 4 doses in total). Before initiating the therapy, biopsy and plasma samples are taken from patients. For longitudinal study, plasma is then taken at 3, 12 and 24 weeks after treatment initiation.

### 7.2. L-Kynurenine Quantification in Plasma Using Enzyme Immuno Assay

L-Kynurenine is then quantified in plasma using an enzyme immuno assay in which the analyte is conjugated to proteins present in the plasma. Because the murine antibody used is raised against L-Kynurenine conjugated to BSA, the proteins in the sample (which is human) do not have to be precipitated (unlike in a murine sample, where endogenous murine immunoglobulins would lead to false positives).

It is in this context surprising that a murine antibody raised against a conjugate consisting of L-Kynurenine and BSA is capable of detecting, in a human sample, different conjugates consisting of L-Kynurenine and different respective proteins present in the plasma, with a sufficient degree of specificity.

Antibody targeting L-Kynurenine-BSA conjugate is affine and specific enough to recognize L-Kynurenine bound to a plasma protein. Briefly, 100 μl of plasma and standards solutions are subjected to derivatization using 100 μl of carbodiimide (EDC) and N-HydroxySuccinimide solubilized in MES buffer (0.3M, pH=6.3) over a 1 hour period under agitation (400 rpm) at 37° C. Details of the derivatization and the conjugation are disclosed in patent application No GB 13 22 538, the content of which is fully incorporated herein by reference. HRP-Kynurenine tracer and a murine anti-L-Kynurenine monoclonal antibody are added to the solution at a final concentration at 0.3 μg/ml and 1 μg/ml respectively.

The latter solution is applied by mean of 200 μl per well on a maxisorp ELISA plate previously coated with unconjugated anti-mouse IgG immunoglobulin. The plate is incubated for 1.30 hour at 37° C. and reaction is revealed using Tetramethylbenzidine. Coloration is monitored at 450 nm with a spectrophotometer.

### 7.3. L-Kynurenine Detection in Tumour Specimens by Immunohistochemistry

Tumours samples are taken from patients suffering from advanced metastatic melanoma before initiation of Ipilimumab therapy. Experimentally, paraffin embedded sections are deparafinized using successive bath of Xylene and Ethanol. Sections are then subjected to antigen retrieval with citrate buffer pH=6 (Dako) for 20 minutes at 95° C. Sections are washed in TBS before incubation with methanol containing 0.03% of hydrogen peroxide to block endogenous peroxydase. After two washes, sections are saturated in antibody diluent (Dako) plus 5% of BSA (Sigma-Aldrich) for 30 minutes at room temperature.

Anti L-Kynurenine mAb (3D4-F2) is then added at 0.01 mg/ml, in the presence of 2% of normal goat serum, and incubated overnight at 4° C. Sections are washed three times in TBS, and incubated for 30 minutes with envision system (dextran polymer grafted with anti mouse IgG conjugated with HRP, Dako) at room temperature. Sections are washed three times before revelation with DAB (Dako) for 10 minutes at room temperature.

Sections are rinsed, subjected to hematoxylin, dehydrated and mounted in DPX mountant media (Sigma-Aldrich). Pictures are obtained after a systematic scan of all cores (Hamamatsu, Nanozzomer). Quantification is performed according to the following grades:

0: No staining
1: Weak staining
2: Intermediate staining
3: Strong staining

### 8. Results

Experiments demonstrate that anti-CTLA4 therapy induced an increase in L-Kynurenine level in plasma compared to patients who received only chemotherapy. Increase is observed at early time points after treatment initiation and is maintained over the time of observation.

Also, patients harboring better clinical response (assessed by radiological imaging) following Ipilimumab based regimen displayed higher amount of L-Kynurenine in plasma.

The concentration of L-Kynurenine (which is a metabolite of the Kynurenine pathway) is thus predictive for the therapeutic efficacy of the drug anti-CTLA4 in the treatment of tumours. Experiments demonstrate that only a fraction of melanoma patients displayed Kynurenine positivity

Also, patients harboring better clinical response (assessed by radiological imaging) following Ipilimumab based regimen are in most of the case highly positive for L-Kynurenine.

The L-Kynurenine positivity in tumour biopsy (which is a metabolite of the Kynurenine pathway) is thus predictive for the therapeutic efficacy of the drug anti-CTLA4 in the treatment of tumours.

### 9. Kynurenine Concentration in a Mice Colorectal Cancer Model According to the Response to Anti-CTLA4

Mice were exposed to anti-CTLA4 (clone UC10-4F10, 100 μg/mouse, Day 3, 6 and 9, ip) and bleedings from the tail vein were performed at different time. Kynurenine measurements from plasma were performed by means of ELISA and revealed that mice rejecting the tumor upon anti-CTLA4 mAb treatment display higher production of Kynurenine seven (7) days after tumor cells inoculation when compared to responding (strong delay in tumor growth when compared to vehicle treated mice) and not responding mice (weak delay in tumor growth when compared to vehicle treated mice).

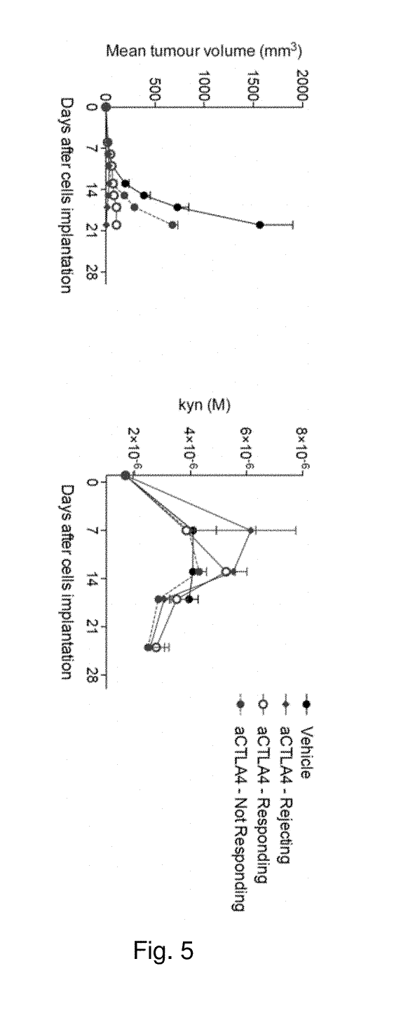

These results show that Kynurenine overproduction upon anti-CTLA4 antibody exposure is a good predictor of response to anti-CTLA4, strongly suggesting that Kynurenine itself could be critical at tuning the host immune response against the tumor. Results are shown in FIG. 5.

### REFERENCES

Duraiswamy et al, Cancer Res; 73(23) Dec. 1, 2013
Ott et al, Clin Cancer Res Oct. 1, 2013, 19:5300-5309
Topalian et al., N Engl J Med 2012; 366:2443-2454
Bessede et al, Nature 511, 184-190 (10 Jul. 2014)
Opitz et al, Nature 2011; 478:197-203.

1-21. (canceled)

22. A method of predicting the therapeutic efficacy of at least one therapy approach involving an agent that targets an immune-checkpoint pathway in the treatment of a neoplastic disease in a patient by means of an immunoassay, which method comprises the following steps:
a) determining the presence or concentration of at least one metabolite of the Kynurenine pathway in a patient sample, and
b) concluding, from step a), whether the at least one therapy approach will be therapeutically effective in the treatment of the neoplastic disease.

23. A method of treating a neoplastic disease in a patient, which method comprises the following steps:
a) determining the presence or concentration of at least one metabolite of the Kynurenine pathway in a patient sample by means of an immunoassay, and
b) dependent on the result of step a), applying, in the patient, one or more therapy approaches involving an agent that targets an immune-checkpoint pathway.

24. The method of claim 22, wherein the presence, absence or concentration of at least one metabolite of the Kynurenine pathway is predictive for the efficacy of said therapy approach.

25. The method of claim 22, wherein the method avoids side effects in patients not responding on treatment with said agent that targets an immune-checkpoint pathway.

26. The method of claim 22, wherein
a) the presence or a high concentration of at least one metabolite of the Kynurenine pathway is predictive of good efficacy of said therapy approach, and/or
b) the absence or low concentration of at least one metabolite of the Kynurenine pathway is predictive of poor efficacy of said therapy approach.

27. The method of claim 26, wherein the high concentration of at least one metabolite of the Kynurenine pathway means a higher concentration thereof compared to a normal concentration of a healthy subject.

28. The method of claim 22, wherein the agent that targets the immune-checkpoint pathway is a modulator, inhibitor, antagonist and/or binder of CTLA4, OX40, PD1, PDL1, Lag3, B7-H3, B7-H4, IDO1, IDO2, TDO2 and/or TIM3.

29. The method of claim 22, wherein the agent that targets the immune-checkpoint pathway is at least one selected from the group consisting of:
a monoclonal antibody (murine, chimeric, humanized, human)
a fragment or derivative thereof (e.g., Fab, Fab2, scFv)
a new antibody format
a fusion peptide comprising at least one domain capable of binding an enzyme and/or a metabolite of the kynurenine pathway
an antibody mimetic,
an aptamer, and/or
a small molecule antagonist.

30. The method of claim 22, wherein the method comprises in step a), the determination of the concentration of two or more metabolites of the Kynurenine pathway, whereupon a logical or arithmetical operation is made based on the determined concentrations, the result of which is then taken as a basis for the decision made in step b).

31. The method of claim 22, wherein the presence or concentration of at least one metabolite in the patient sample is determined by immunohistochemistry, ELISA, EIA and/or immunofluorescence.

32. The method of claim 22, wherein the metabolite of the kynurenine pathway in the sample is derivatized, prior to detection, by conjugating it to a carrier molecule.

33. The method of claim 32, wherein the carrier molecules added to the sample.

34. The method of claim 32, wherein the carrier molecule is a naturally occurring carrier molecule different from the serum proteins that are part of the sample.

35. The method of claim 22, wherein a detection immunoligand is used in the immunoassay, said immunoligand being created against a complex consisting of a metabolite of the Kynurenine pathway and a carrier.

36. The method of claim 35, wherein the detection immunoligand specifically binds to the metabolite-carrier complex and has been created by a method comprising the steps of:
a) conjugating the Kynurenine pathway metabolite, in isolated form, to a carrier molecule to obtain an immunogenic conjugate,
b) carrying out an immunization experiment with said immunogenic conjugate, and
c) obtaining, directly or indirectly, detection antibodies from said experiment that specifically bind to the metabolite-carrier complex and/or to the metabolite.

37. The method of claim 35, wherein the detection immunoligand is created by a method comprising the steps of:
a) exposing said analyte, or a metabolite-carrier complex to a library of immunoligands, and
b) screening said library for detection immunoligands that specifically bind to the metabolite-carrier complex and/or to the analyte.
